SEQUENCE LISTING (1 sequence)

SEQ ID NO: 1 - protein, 672 aa, synthetic construct
SLVSLLSVLLMGCVAETGTQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPI

(19) United States
(12) Patent Application Publication
(10) Pub. No.: US 2026/0139008 A1
(43) Pub. Date: May 21, 2026

(54) METHODS OF TREATING LUNG CANCER AND OTHER CANCERS BY RECOMBINANT SARS-COV-2 SPIKE S1

(71) Applicant: Rush University Medical Center, Chicago, IL (US)

(72) Inventor: Kalipada PAHAN, Skokie, IL (US)

(73) Assignee: Rush University Medical Center, Chicago, IL (US)

(21) Appl. No.: 18/706,910

(22) PCT Filed: Nov. 1, 2022
(86) PCT No.: PCT/US2022/079091

§ 371 Date: May 2, 2024

Related U.S. Application Data
(60) Provisional application No. 63/274,643, filed on Nov. 2, 2021.

(51) Int. Cl. C07K 14/005; A61K 9/00; A61K 38/00; A61P 35/00

(52) U.S. Cl. CPC C07K 14/005; A61K 9/0043; A61P 35/00; A61K 38/00; C12N 2770/20022; C12N 2770/20033

(57) ABSTRACT

Described herein is a novel method of treating cancer in a patient in need thereof, comprising administering a therapeutically effective amount of SARS-CoV-2 Spike 51 polypeptide.

### CROSS-REFERENCE TO RELATED APPLICATION

This application claims the benefit of U.S. Provisional Application No. 63/274,643, filed Nov. 2, 2021, the contents of which are hereby incorporated by reference.

### STATEMENT REGARDING SEQUENCE LISTING

The Sequence Listing associated with this application is provided in XML format in lieu of paper copy, and is hereby incorporated by reference into the specification. The name of the XML file containing the Sequence Listing is “42960-374014_ST26.” The text file is 2.59 KB, was created on Nov. 1, 2022, and is being submitted electronically, concurrent with the filing of this specification.

### TECHNICAL FIELD

Described herein is a novel method of treating cancer comprising administration of a recombinant SARS-CoV-2 spike S1 protein.

### BACKGROUND OF THE INVENTION

According to the Centers for Disease Control and Prevention, cancer is the number two cause of death in the USA, second only to heart disease [1]. Ironically, among all cancers, lung cancer is by far the leading cause of cancer death in which more than 130,000 people die each year in the USA. In fact, more people die of lung cancer than of colon, breast and prostate cancers combined, approximately amounting to 25% of all cancer deaths in the USA [2]. The five-year survival rate for lung cancer patients (22%) is also significantly lower than other cancers [3].

Therefore, understanding molecular mechanisms and developing an effective therapeutic approach for lung cancer are of paramount importance. Interestingly, the renin-angiotensin system plays an important role in lung tumor progression or metastasis [4]. It has been shown that the low expression of angiotensin-converting enzyme 2 (ACE2) is associated with tumor grade in lung cancer and that overexpression of ACE2 suppresses the invasion and angiogenesis of non-small cell lung cancer (NSCLC) [5,6]. Therefore, stimulation of ACE2 may be an important mechanism to control lung cancer growth.

Through the recent COVID-19 pandemic, SARS-CoV-2 was shown to be a unique molecule that activates and stimulates the ACE2 signaling pathway [7-10]. The spike S1 protein of SARS-CoV-2 binds to the ACE2 receptor to enter and infect human cells [8-11]. Thus, SARS-CoV-2 spike S1 may provide a possible therapeutic potential for cancers that overexpress ACE2 receptor.

### INCORPORATION BY REFERENCE

All publications, patents, and patent applications mentioned in this specification are herein incorporated by reference with regard to their background teachings to the same extent as if each individual publication, patent, or patent application was specifically and individually indicated to be incorporated by reference.

### SUMMARY OF THE INVENTION

One embodiment described herein is a method of treating cancer comprising administering to a patient in need thereof, a therapeutically effective amount of a recombinant SARS-CoV-2 Spike S1 or a biologically active fragment thereof. In one aspect, the recombinant SARS-CoV-2 Spike S1 or a biologically active fragment thereof comprises the amino acid sequence of SEQ ID NO. 1.

In another aspect, the recombinant SARS-CoV-2 Spike S1 or a biologically active fragment thereof is administered intranasally, subcutaneously, or intravenously.

In one aspect, the recombinant SARS-CoV-2 Spike S1 or a biologically active fragment thereof is administered intranasally

In another aspect, the therapeutically effective amount comprises about 1 to about 50 ng/mL. In one aspect, the therapeutically effective amount comprises about 50 ng/kg body weight. In another aspect, the therapeutically effective amount comprises about 1 ng/mL. In another aspect, the therapeutically effective amount comprises about 5 ng/mL. In one aspect, the therapeutically effective amount comprises about 10 ng/mL.

In another aspect, the cancer comprises lung cancer, kidney cancer or heart cancer. In one aspect, the cancer comprises lung cancer.

In another aspect, the method further comprising administration of one or more additional therapeutic agents.

Another embodiment described herein is an isolated SARS-CoV-2 Spike S1 polypeptide comprising the amino acid sequence of SEQ ID NO: 1. In one aspect is a pharmaceutical composition comprising the polypeptide described herein and a pharmaceutically acceptable carrier.

### BRIEF DESCRIPTION OF THE DRAWINGS

The features of the present disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1: Effect of recombinant SARS-CoV-2 spike S1 on the survival of human A549 lung cancer cells. A549 cells were treated with spike S1 protein for 24 h under serum-free condition followed by monitoring cell death by LDH release (FIG. 1A) and MTT (FIG. 11B). Annexin V and PI FACS double staining was also performed (FIG. 1C). Quantitative analysis of percent apoptotic cells is presented (FIG. 1D). Values are presented as mean±SD of three independent experiments. *p<0.05; ** p<0.01.

FIG. 2: Recombinant SARS-CoV-2 spike S1 induces apoptosis in human A549 lung cancer cells. A549 cells were treated with different doses of spike S1 protein for 12 h under serum-free condition followed by monitoring apoptosis by TUNEL (FIG. 2A). TUNEL positive cells were counted in 10 varied images per group and plotted as percent of total cells (FIG. 2B). FIG. 2C) Cells were immunoblotted for apoptosis-related molecules (BAD, caspase 3 and cleaved caspase 3). Actin was run as a loading control. Bands were scanned and values (FIG. 2D, BAD/Actin; FIG. 2E, cleaved caspase 3/Actin; F, caspase 3/Actin) presented as relative to control. FIG. 2G) Cells were immunoblotted for survival-related molecule (Bcl2). Actin was run as a loading control. FIG. 2H) Bands were scanned and values (Bcl2/Actin) presented as relative to control. Results are mean+SD of three different experiments. *p<0.05; ** p<0.01; *** p<0.001. Scale bar=22 μm.

FIG. 3: Spike S1-mediated death of human A549 lung cancer cells depends on ACE2 receptor. A549 cells were treated with 5 ng/ml spike S1 protein in the presence or absence of neutralizing antibodies against spike S1 (0.5 μg/ml) under serum-free condition. After 24 h, cell viability was monitored by LDH release (FIG. 3A) and MTT (FIG. 3B). Control A549 cells were immunostained for ACE2 (FIG. 3C). DAPI was used to stain nuclei. Cells were treated with 5 ng/ml spike S1 protein in the presence or absence of neutralizing antibodies against ACE2 (0.5 μg/ml) under serum-free condition. After 24 h, cell viability was monitored by LDH release (FIG. 3D) and MTT (FIG. 3E). Results are mean+SD of three different experiments. *p<0.05; ** p<0.01; ***p<0.001. Scale bar=10 μm.

FIG. 4: Effect of recombinant SARS-CoV-2 spike S1 on the survival of human H1299 and H358 lung cancer cells. H1299 (FIGS. 4A & 4C) and H358 (FIGS. 4B & 4D) cells were treated with spike S1 protein for 24 h under serum-free condition followed by monitoring cell death by LDH release (FIGS. 4A & 4B) and MTT (FIGS. 4C & 4D). Results are mean+SD of three different experiments. *p<0.05; ** p<0.01; ***p<0.001.

FIG. 5: Intranasal administration of recombinant SARS-CoV-2 spike S1 causes regression of lung tumor in NNK-challenged female A/J mice. The experimental design is illustrated for NNK-induced lung cancer in A/J mice (FIG. 5A). Briefly, female A/J mice (5-6 week old) received two intraperitoneal (i.p.) injections of NNK (50 mg/kg body weight) one week apart. Tumor development was analyzed after 26 weeks of NNK intoxication. Mice were treated with spike S1 (50 ng/mouse/2 d) intranasally on alternate days starting from 22 weeks of NNK insult for 4 weeks followed by sacrificing mice on 26 weeks. Representative lung appearance in different groups of mice (FIG. 5B). Lung sections were stained for H&E (FIG. 5C). The histological tumor area was quantified in a 4× field as a percent of control (FIG. 5D). The number of lung lesions is shown in different groups of mice (E). Results are mean+SD of 5 mice per group. *p<0.05; ** p<0.01; ***p<0.001. Scale bar=20 μm.

FIG. 6: Intranasal administration of recombinant SARS-CoV-2 spike S1 induces apoptosis in lung tumors of NNK-insulted female A/J mice. Female A/J mice (5-6 week old) received two intraperitoneal (i.p.) injections of NNK (50 mg/kg body weight) one week apart. Mice were treated with spike S1 (50 ng/mouse/2 d) intranasally on alternate days starting from 22 weeks of NNK-insult for 4 weeks followed by sacrificing mice on 26 weeks. Tumor tissue sections were labeled for TUNEL (FIG. 6A) followed by counting of TUNEL-positive cells in two sections (two images per section) of each of five mice per group (FIG. 6B). Results are mean+SD of 5 mice per group. *p<0.05; ** p<0.01; ***p<0.001. Scale bar=40 μm.

### DETAILED DESCRIPTION OF THE DISCLOSURE

The following description and examples illustrate embodiments of the present disclosure in detail.

It is to be understood that the present disclosure is not limited to the particular embodiments described herein and as such may vary. Those of skill in the art will recognize that there are variations and modifications of the present disclosure, which are encompassed within its scope.

All terms are intended to be understood as they would be understood by a person skilled in the art. Unless defined otherwise, all technical and scientific terms used herein have the same meaning as commonly understood by one of ordinary skill in the art to which the disclosure pertains.

The section headings used herein are for organizational purposes only and are not to be construed as limiting the subject matter described.

Although various features of the disclosure may be described in the context of a single embodiment, the features may also be provided separately or in any suitable combination. Conversely, although the present disclosure may be described herein in the context of separate embodiments for clarity, the present disclosure may also be implemented in a single embodiment. The following definitions supplement those in the art and are directed to the current application and are not to be imputed to any related or unrelated case, e.g., to any commonly owned patent or application. Although any methods and materials similar or equivalent to those described herein may be used in the practice for testing of the present disclosure, the preferred materials and methods are described herein. Accordingly, the terminology used herein is for the purpose of describing particular embodiments only, and is not intended to be limiting.

In this application, the use of the singular includes the plural unless specifically stated otherwise. It must be noted that, as used in the specification, the singular forms “a,” “an” and “the” include plural referents unless the context clearly dictates otherwise.

In this application, the use of “or” means “and/or” unless stated otherwise. The terms “and/or” and “any combination thereof” and their grammatical equivalents as used herein, may be used interchangeably. These terms may convey that any combination is specifically contemplated. Solely for illustrative purposes, the following phrases “A, B, and/or C” or “A, B, C, or any combination thereof” may mean “A individually; B individually; C individually; A and B; B and C; A and C; and A, B, and C.” The term “or” may be used conjunctively or disjunctively, unless the context specifically refers to a disjunctive use.

Furthermore, use of the term “including” as well as other forms, such as “include,” “includes,” and “included,” is not limiting.

Reference in the specification to “some embodiments,” “some aspects,” “an embodiment,” “an aspect,” “another aspect”, “one embodiment,” “one aspect” or “other embodiments” or “other aspects” means that a particular feature, structure, or characteristic described in connection with the embodiments is included in at least some embodiments, but not necessarily all embodiments, of the present disclosures.

As used in this specification and the claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification may be implemented with respect to any method or composition of the disclosure, and vice versa. Furthermore, compositions of the present disclosure may be used to achieve methods of the present disclosure.

The term “about” or “approximately” means within an acceptable error range for the particular value as determined by one of ordinary skill in the art, which will depend in part on how the value is measured or determined, i.e., the limitations of the measurement system. For example, “about” may mean within 1 or more than 1 standard deviation, per the practice in the art. Alternatively, “about” may mean a range of up to 20%, up to 10%, up to 5%, or up to 1% of a given value. In another example, the amount “about 10” includes 10 and any amounts from 9 to 11. In yet another example, the term “about” in relation to a reference numerical value may also include a range of values plus or minus 10%, 9%, 8%, 7%, 6%, 5%, 4%, 3%, 2%, or 1% from that value. Alternatively, particularly with respect to biological systems or processes, the term “about” may mean within an order of magnitude, preferably within 5-fold, and more preferably within 2-fold, of a value. Where particular values are described in the application and claims, unless otherwise stated the term “about” meaning within an acceptable error range for the particular value should be assumed.

“Administering” is referred to herein as providing one or more compositions or therapies as described herein to a patient or a subject. By way of example and not limitation, composition administration, e.g., injection, may be performed by intravenous (i.v.) injection, sub-cutaneous (s.c.) injection, intradermal (i.d.) injection, intraperitoneal (i.p.) injection, or intramuscular (i.m.) injection, or intranasal. One or more such routes may be employed. Parenteral administration may be, for example, by bolus injection or by gradual perfusion over time. Alternatively, or concurrently, administration may be by the oral route or intranasally. Additionally, administration may also be by surgical deposition of a bolus or pellet of cells, or by medical device.

As used herein, the term “amount” refers to “an amount effective” or “therapeutically effective amount” of a recombinant SARS-CoV-2 Spike S1 protein, to achieve a beneficial or desired prophylactic or therapeutic result, including clinical results. A “therapeutically effective amount” of a recombinant SARS-CoV-2 Spike S1 protein may vary according to factors such as the disease state, age, sex, and weight of the individual. A therapeutically effective amount is also one in which any toxic or detrimental effects protein are outweighed by the therapeutically beneficial effects. The term “therapeutically effective amount” includes an amount that is effective to “treat” a subject (e.g., a patient). When a therapeutic amount is indicated, the precise amount of the protein and/or compositions of the protein in the present disclosure to be administered may be determined by a physician with consideration of individual differences in age, weight, tumor size, extent of infection or metastasis, and condition of the patient (subject).

As used herein, the term “cancer” relates generally to a class of diseases or conditions in which abnormal cells divide without control and may invade nearby tissues.

As used herein, the term “cancerous cell,” “cancer cell,” “tumor cell” or variant thereof refers to an individual cell of a cancerous growth or tissue. A tumor refers generally to a swelling or lesion formed by an abnormal growth of cells, which may be benign, pre-malignant, or malignant. Most cancers form tumors, but some, e.g., leukemia, do not necessarily form tumors. For those cancers that form tumors, the terms cancer (cell) and tumor (cell) are used interchangeably. The amount of a tumor in an individual is the “tumor burden” which may be measured as the number, volume, or weight of the tumor.

The term “composition” as used herein is intended to encompass a product comprising specific ingredients in specified amounts, as well as any product which results, directly or indirectly, from combination of the specified ingredients in the specified amounts. By “pharmaceutically acceptable” it is meant the carrier, diluent or excipient must be compatible with the other ingredients of the formulation, including the vectors described herein, and not deleterious to the recipient thereof. A “pharmaceutically acceptable carrier” is any carrier which is relatively non-toxic and innocuous to a patient at concentrations consistent with effective activity of the active ingredient so that any side effects ascribable to the carrier do not vitiate the beneficial effects of the active ingredient.

“Patient” or “subject” as used herein refers to a mammalian subject diagnosed with or suspected of having a cancer. Exemplary patients may be humans, apes, dogs, pigs, cattle, cats, horses, goats, sheep, rodents and other mammalians that may benefit from the therapies disclosed herein. Exemplary human patients may be male and/or female. “Patient in need thereof” or “subject in need thereof” is referred to herein as a patient diagnosed with or suspected of having a disease or disorder, for instance, but not restricted to cancer. For example, lung cancer, kidney cancer, or cancers in which there is overexpression of ACE2 receptor.

As used herein, the term “substantially” refers to a quantity, level, value, number, frequency, percentage, dimension, size, amount, weight or length that is 80%, 85%, 90%, 91%, 92%, 93%, 94%, 95%, 96%, 97%, 98%, 99% or higher of a reference quantity, level, value, number, frequency, percentage, dimension, size, amount, weight or length. In one embodiment, “substantially the same” refers to a quantity, level, value, number, frequency, percentage, dimension, size, amount, weight or length that produces an effect, e.g., a physiological effect, that is approximately the same as a reference quantity, level, value, number, frequency, percentage, dimension, size, amount, weight or length.

As used herein, the term “treatment”, “treating”, or its grammatical equivalents refers to obtaining a desired pharmacologic and/or physiologic effect. In some embodiments, the effect is therapeutic, i.e., the effect partially or completely cures a disease and/or condition. In some embodiments, the term “treating” may include inhibiting or “preventing” a disease or a condition, including cancer.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The present disclosure describes novel methods of treating cancer comprising administering a recombinant SARS-CoV-2 Spike S1 protein.

The genome of SARS-CoV-2 was determined, as was the crystal structure of the viral envelope spike glycoprotein (S protein), responsible for attachment and fusion with human cells and thus host-to-host transmission. S protein is proteolytically cleaved into S1 and S2 subunits prior to infection. S1 binds to a receptor on the target cell surface known as angiotensin converting enzyme 2 (ACE2), which is thought to initiate a series of conformational changes in S2 which facilitates viral fusion and the initiation of infection. Because of the low expression of angiotensin-converting enzyme 2 (ACE2) is associated with tumor grade in lung cancer and that overexpression of ACE2 suppresses the invasion and angiogenesis of non-small cell lung cancer (NSCLC) [5,6], stimulation of the ACE2 receptor may play a role in cancer growth.

Thus one embodiment described herein is a method of treating cancer comprising administering to a patient in need thereof, a therapeutically effective amount of a recombinant SARS-CoV-2 Spike S1 protein, or a biologically active fragment thereof. In one aspect, the the recombinant SARS-CoV-2 Spike S1 or a biologically active fragment thereof comprises the amino acid sequence of SEQ ID NO. 1.

(SEQ ID NO: 1)
SLVSLLSVLLMGCVAETGTQCVNLTTRTQLPPAYTNSFTRGVYYPDKVFR
SSVLHSTQDLFLPFFSNVTWFHAIHVSGTNGTKRFDNPVLPFNDGVYFAS
TEKSNIIRGWIFGTTLDSKTQSLLIVNNATNVVIKVCEFQFCNDPFLGVY
YHKNNKSWMESEFRVYSSANNCTFEYVSQPFLMDLEGKQGNFKNLREFVF
KNIDGYFKIYSKHTPINLVRDLPQGFSALEPLVDLPIGINITRFQTLLAL
HRSYLTPGDSSSGWTAGAAAYYVGYLQPRTFLLKYNENGTITDAVDCALD
PLSETKCTLKSFTVEKGIYQTSNFRVQPTESIVRFPNITNLCPFGEVFNA
TRFASVYAWNRKRISNCVADYSVLYNSASFSTFKCYGVSPTKLNDLCFTN
VYADSFVIRGDEVRQIAPGQTGKIADYNYKLPDDFTGCVIAWNSNNLDSK
VGGNYNYLYRLFRKSNLKPFERDISTEIYQAGSTPCNGVEGENCYFPLQS
YGFQPTNGVGYQPYRVVVLSFELLHAPATVCGPKKSTNLVKNKCVNFNFN
GLTGTGVLTESNKKFLPFQQFGRDIADTTDAVRDPQTLEILDITPCSFGG
VSVITPGTNTSNQVAVLYQDVNCTEVPVAIHADQLTPTWRVYSTGSNVFQ
TRAGCLIGAEHVNNSYECDIPI.

In one aspect described herein, polynucleotides encoding one or more of the proteins contemplated herein are provided. As used herein, “isolated polynucleotide” refers to a polynucleotide that has been purified from the sequences which flank it in a naturally-occurring state, e.g., a DNA fragment that has been removed from the sequences that are normally adjacent to the fragment. An “isolated polynucleotide” also refers to a complementary DNA (cDNA), a recombinant DNA, or other polynucleotide that does not exist in nature and that has been made by the hand of man. As used herein, the terms “polynucleotide” or “nucleic acid” refers to messenger RNA (mRNA), RNA, genomic RNA (gRNA), plus strand RNA (RNA(+)), minus strand RNA (RNA(−)), complementary DNA (cDNA) or recombinant DNA. Polynucleotides include single and double stranded polynucleotides. One aspect described herein is an isolated nucleic acid molecule encoding any of the proteins or polypeptides described herein.

Polynucleotides of the disclosure include polynucleotides or variants having at least about 50%, 55%, 60%, 65%, 70%, 75%, 80%, 85%, 90%, 95%, 96%, 97%, 98%, 99% or 100% sequence identity to any of the reference sequences described herein (see, e.g., Sequence Listing), typically where the variant maintains at least one biological activity of the reference sequence. In various illustrative embodiments, the present disclosure contemplates, in part, polynucleotides comprising expression vectors, viral vectors, and transfer plasmids, and compositions, and cells comprising the same.

An example of an algorithm that is suitable for determining percent sequence identity and sequence similarity is the BLAST algorithm, which is described in Altschul et al., 1977, Nuc. Acids Res. 25:3389-3402. BLAST is used, with the parameters described herein, to determine percent sequence identity for the nucleic acids and proteins of the invention. Software for performing BLAST analysis is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA, 89:10915 (1989)) alignments (B) of 50, expectation (E) of 10, M=5, N=−4, and a comparison of both strands.

The BLAST algorithm also performs a statistical analysis of the similarity between two sequences (see, e.g., Karlin & Altschul, Proc. Nat'l. Acad. Sci. USA, 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

As used herein, the terms “polynucleotide variant” and “variant” and the like refer to polynucleotides displaying substantial sequence identity with a reference polynucleotide sequence or polynucleotides that hybridize with a reference sequence under stringent conditions that are defined hereinafter. These terms include polynucleotides in which one or more nucleotides have been added or deleted, or replaced with different nucleotides compared to a reference polynucleotide. In this regard, it is well understood in the art that certain alterations inclusive of mutations, additions, deletions and substitutions may be made to a reference polynucleotide whereby the expressed altered polynucleotide retains the biological function or activity of the reference polynucleotide.

Polynucleotides may be prepared, manipulated and/or expressed using any of a variety of well-established techniques known and available in the art. In order to express a desired polypeptide, a nucleotide sequence encoding the polypeptide, may be inserted into an appropriate vector. Examples of vectors are plasmid, autonomously replicating sequences, and transposable elements. Additional exemplary vectors include, without limitation, plasmids, phagemids, cosmids, artificial chromosomes such as yeast artificial chromosome (YAC), bacterial artificial chromosome (BAC), or P1-derived artificial chromosome (PAC), bacteriophages such as lambda phage or M13 phage, and animal viruses. Examples of categories of animal viruses useful as vectors include, without limitation, retrovirus (including lentivirus), adenovirus, adeno-associated virus, herpesvirus (e.g., herpes simplex virus), poxvirus, baculovirus, papillomavirus, and papovavirus (e.g., SV40). Examples of expression vectors are pClneo vectors (Promega) for expression in mammalian cells; pLenti4N5-DEST™ pLenti6N5-DEST™, and pLenti6.2N5-GW/lacZ (Invitrogen) for lentivirus-mediated gene transfer and expression in mammalian cells. In some embodiments, the coding sequences of the polypeptides or proteins disclosed herein may be ligated into such expression vectors for the expression of peptides in mammalian cells. One aspect described herein is an expression vector comprising an isolated nucleic acid of any of the polypeptides described herein.

Polypeptides of the disclosure include polypeptides having at least about 50%, 60%, 65%, 70%, 75%, 85%, 90%, 95%, 98%, or 99% amino acid identity thereto. Peptides of the disclosure include variants having at least about 50%, 55%, 60%, 65%, 70%, 75%, 80%, 85%, 90%, 95%, 96%, 97%, 98%, 99% or 100% sequence identity to any of the reference sequences described herein (see, e.g., Sequence Listing), typically where the variant maintains at least one biological activity of the reference sequence. Polypeptides include “peptide fragments.” Peptide fragments refer to a peptide, which may be monomeric or multi-meric that has an amino-terminal deletion, a carboxyl-terminal deletion, and/or an internal deletion or substitution of a naturally-occurring or recombinantly-produced polypeptide. In one aspect described herein, the recombinant SARS-CoV2 Spike S1 protein is a recombinant polypeptide.

In certain embodiments, a peptide fragment may comprise an amino acid chain at least 5 to about 500 amino acids long. It will be appreciated that in certain embodiments, fragments are at least 5, 6, 7, 8, 9, 10, 11, 12, 13, 14, 15, 16, 17, 18, 19, 20, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, or 100 amino acids long.

A “host cell” includes cells transfected, infected, or transduced in vivo, ex vivo, or in vitro with a recombinant vector or a nucleic acid, polynucleotide, peptide or polypeptide of the disclosure. Host cells may include packaging cells, producer cells, and cells infected with viral vectors. In some embodiments, host cells infected with viral vector of the disclosure are administered to a subject in need of therapy. In certain embodiments, the term “target cell” is used interchangeably with host cell and refers to transfected, infected, or transduced cells of a desired cell type.

Exemplary cancers contemplated herein include, but are not limited to, lung cancer, heart cancer, or kidney cancer. Thus, in one aspect the cancer comprises lung cancer. In another aspect, the cancer comprises any cancer in which ACE2 is expressed.

According to another aspect of the present disclosure, pharmaceutical compositions of any of the polypeptides described herein are provided.

Appropriate pharmaceutical compositions comprising the polypeptides contemplated herein, and are based partly on the specific tissues, and cell types involved. Pharmaceutical compositions appropriate for the cells of the instant disclosure may be thus be formulated according to any means know in the art. (See for example: Remington's Pharmaceutical Sciences, 15th Edition, chapter 33; Gagliardi et al., 2021; or Hammond et al., 2021).

The pharmaceutical compositions for the administration of the polypeptides of this disclosure may conveniently be presented in unit dosage form and may be prepared by any of the methods well known in the art of pharmacy. All methods include the step of bringing the active ingredient into association with a carrier which constitutes one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition, the active polypeptides is included in an amount sufficient to produce the desired effect upon the process or condition of diseases.

The polypeptides or compositions described herein may be administered with a pharmaceutically-acceptable carrier using any effective conventional dosage unit forms, including, for example, orally, intranasally or the like. In some aspects, the polypeptides or compositions may be administered intranasally.

The compositions of the disclosure may also contain other conventional pharmaceutically acceptable compounding ingredients, generally referred to as carriers or diluents, as necessary or desired. Any of the compositions of this disclosure may be preserved by the addition of an antioxidant such as ascorbic acid or by other suitable preservatives. Conventional procedures for preparing such compositions in appropriate dosage forms may be utilized.

In another aspect, the subject is a human or a patient. In another aspect, the effective amount is any amount required to demonstrate a therapeutic effect. The therapeutically effective dosage of the cells or pharmaceutical compositions of the instant disclosure may readily be determined for treatment of each desired indication.

The therapeutically effective amount of the polypeptides of this disclosure may readily be determined for treatment of each desired indication. The amount of the active ingredient (e.g., polypeptides) to be administered in the treatment of one of these conditions may vary widely according to such considerations as the particular polypeptide and dosage unit employed, the mode of administration, the period of treatment, the age and sex of the patient treated, and the nature and extent of the condition treated.

In one aspect, therapeutically effective amount of the SARS-CoV-2 Spike S1 polypeptide to be administered may generally range from about 1 ng/mL to about 50 ng/mL, about 1 ng/mL to about 40 ng/mL, about 1 ng/mL to about 30 ng/mL, about 1 ng/mL to about 20 ng/mL, about 1 ng/mL to about 10 ng/mL, about 1 ng/mL, about 2 ng/mL, about 3 ng/mL, about 4 ng/mL, about 5 ng/mL, about 6 ng/mL, about 7 ng/mL, about 8 ng/mL, about 9 mg/mL or about 10 ng/mL.

In another aspect, therapeutically effective amount of the SARS-CoV-2 Spike S1 polypeptide to be administered may generally range from about 10 to about 100 ng/kg body weight, about 20 to about 90 ng/kg body weight, about 30 to about 80 ng/kg body weight, about 40 to about 75 ng/kg body weight, about 45 to about 65 ng/kg body weight, about 45 ng/kg body weight, about 50 ng/kg body weight or about 60 ng/kg body weight.

A unit dosage may contain from about 0.05 mg to about 500 mg of active ingredient, and may be administered one or more times per day. The daily dosage for administration by injection, including intravenous, intramuscular, subcutaneous, and intranasally may be from about 0.0001 mg/kg to about 10 mg/kg. The daily intranasal concentration may be that required to maintain a daily dose of from 0.0001 mg/kg to 10 mg/kg.

Furthermore, the unit dosage may be administered multiple times daily, once daily, every 2 days, twice a week, once a week, biweekly, or monthly.

The specific initial and continuing dosage regimen for each patient will vary according to the nature and severity of the condition as determined by the attending diagnostician, the activity of the specific peptide employed, the age of the patient, the diet of the patient, time of administration, route of administration, rate of excretion of the drug, drug combinations, and the like. The desired mode of treatment and number of doses of the polypeptide of the present disclosure may be ascertained by those skilled in the art using conventional treatment tests.

Another aspect described herein is a method of treating cancer comprising administering the polypeptides or pharmaceutical compositions described herein, wherein the polypeptide or composition may be administered in combination with one or more chemotherapeutic agents, targeted inhibitors, immune checkpoint inhibitors, cell therapies, monoclonal antibodies, oncolytic virus therapies, cancer vaccines, or immune system modulators, including but not limited to the full spectrum of compositions and compounds which are known to be active in killing and/or inhibiting the growth of cancer cells.

Chemotherapeutic agents, may include, but are not limited to to cisplatin, carboplatin, camptothecin, indolizino, irinotecan, diflomotecan, exatecan, gimatecan, irinotecan, karenitecin, lurtorecan, rubitecan, silatecan, topotecan

Antibodies may be polyclonal or monoclonal antibodies, humanized or human, that bind to an epitope on any of the cancers described herein. Any suitable antibody targeting the specific cancer contemplated herein may be used. In one aspect is a method of treating cancer comprising administering to a subject in need thereof, an effective amount of the polypeptides or a pharmaceutical composition comprising the polypeptides described herein in combination with one or more antibody. Exemplary antibodies for use with the cells or pharmaceutical composition described herein include rituximab, trastuzumab, ibritumomab, cetuximab, bevacizumab, pantiumumab, ofatumumab, ipilimumab, brentuximab vedotin, pertuzumab, ado-trastuzumab emtansine, obinutuzumab, ramucirumab, pembrolizumab, blinatumomab, nivolumab, dinutuximab, daratumumab, necitumumab, elotuzumab, atezolizumab, olaratumab, avelumab, durvalumab, inotuzumab ozogamicin, tisagenlecleucel, gemtuzamab ozogamicin, axicabtagene ciloleucel, mogamulizumab kpkc, moxetumomab pasudotox-tdfk, cemiplimab-rwlc, polatuzumab vedotinpiiq, enfortumab vedotinejfv, or fam-trastuzumab. Other antibodies not described herein may also be used in combination with the cells or pharmaceutical compositions described herein.

Targeted inhibitors comprise any targeted therapy, including but not limited to, therapies that target a specific gene or protein. These may include targeted therapies specific to a type of cancer. Examples of targeted inhibitors include inhibitors of HER2, BCR-ABL, EGFR, and VEGF, PARP or kinase inhibitors.

In another aspect of the present disclosure the polypeptides or pharmaceutical composition may be administered in combination with one or more additional therapeutic agent. Potential other drugs include but not limited to: chemotherapeutic drugs including but not limited to camptothecin, indolizino, irinotecan, diflomotecan, exatecan, gimatecan, irinotecan, karenitecin, lurtorecan, rubitecan, silatecan, topotecan; targeted inhibitors; and antibodies.

Depending on the individual medicaments utilized in a combination therapy for simultaneous administration, they may be formulated in combination (where a stable formulation may be prepared and where desired dosage regimes are compatible) or the medicaments may be formulated separately (for concomitant or separate administration through the same or alternative routes).

It will be readily apparent to one of ordinary skill in the relevant arts that suitable modifications and adaptations to the compositions, methods, and applications described herein may be made without departing from the scope of any embodiments or aspects thereof. The compositions and methods provided are exemplary and are not intended to limit the scope of any of the specified embodiments. All of the various embodiments, aspects, and options disclosed herein may be combined in any and all variations or iterations. The scope of the compositions, formulations, methods, and processes described herein include all actual or potential combinations of embodiments, aspects, options, examples, and preferences herein described.

### EXAMPLES

### Reagents

Recombinant SARS-CoV-2 spike S1 (14-685) was purchased from Abeomics, San Diego, CA. Recombinant human ACE2 protein (18-739) was purchased from MyBiosource, San Diego, CA. Human lung carcinoma cell lines (A549, H1299 and H358) and F-12K medium were obtained from ATCC, Manassas, VA. Hank's balanced salt solution, RPMI-1640, penicillin, streptomycin, and 0.05% trypsin were bought from Mediatech (Washington, DC). Fetal bovine serum (FBS) was obtained from Atlas Biologicals, Fort Collins, CO. While anti-SARS-CoV-2 spike S1 antibody was bought from BioVision (Milpitas, CA), anti-hACE2 antibody was purchased from R&D Systems (Minneapolis, MN).

### Example 1

### Cell Culture

A549 (human lung carcinoma; KRAS mut; EGFR wt) non-small cell lung cancer (NSCLC) cells were maintained at 37° C. and 5% CO₂ in F12K media, supplemented with 10% FBS, 100 U/mL penicillin, and 100 μg/mL streptomycin. Once cells reached 80% confluence, these were passaged. Cells were washed with phosphate-buffered solution (PBS) and treated with 0.25% Trypsin. Cells were suspended in F12K culture medium and seeded into T75 flasks.

The same procedure was utilized for other cell lines purchased from ATCC:
H1299 (human NSCLC, p53 negative)
H358 (human NSCLC, KRAS mut)
   These cells were cultured in RPMI-1640. Cells at logarithmic phase were used for experiments.

### Example 2

### Cytotoxicity Assays: MTT Assay

The viability of cells was evaluated using the 3-(4,5-dimethylthiazol-2-yl)-2,5-diphenyl tetrazolium bromide (MTT) method with an in vitro toxicology assay kit from Sigma was seeded to measure mitochondrial activity. Cells were seeded in 24-well plates with 500 mL of F12K medium then switched to serum free medium. Before adding MTT, 100 mL was removed to be used for LDH assay. MTT was added to each well for 2 hours according to the protocol outlined by the manufacturer. After removing the supernatant, formazan crystals were dissolved by adding equal volume of solution. At the end of the treatment period, 300 mL of culture medium was removed from each well and 20 mL of MTT solution (5 mg/mL) was added and incubated for 30 min. After distribution to a 96-well plate, absorbance was measured at 595 nm with the Thermo-Fisher Multiskan™ MCC plate reader (Fisher).

### Example 3

### Lactate Dehydrogenase Measurement

The activity of lactate dehydrogenase (LDH) was measured using a lactate dehydrogenase activity assay kit (Sigma). A volume from the MTT assay was used and plated in a 96-well plate. An LDH master mix was prepared and added to each well. The reaction was carried out at room temperature in the dark. The resultant absorbance was measured at 450 nm with the Thermo-Fisher Multiskan™ MCC plate reader (Fisher).

### Example 4

### Fragment End Labeling DNA

Fragmented DNA was detected in situ by the terminal deoxynucleotide transferase (TdT) mediated binding of 3′OH ends of DNA fragments generated in response to apoptotic signals, using a commercially available kit (TdT FragEL DNA Detection Kit) from Sigma (EMD Millipore). Coverslips containing A549 lung adenocarcinoma cells cultured to 70-80% confluence were fixed with chilled methanol (Fisher Scientific, Waltham, MA) for an hour, followed by two brief rinses with sterile PBS. Cover slips were treated with 20 mg/mL proteinase K for 5 min at room temperature and washed in PBS before TdT staining. Samples were equilibrated for 30 min in 1×TdT buffer and washed with PBS prior to terminal deoxynucleotidyl transferase and DAPI (1:10,000, Sigma) staining. After mounting coverslips and drying overnight, slides were visualized using a Bio-Rad (Hercules, CA) MRC1024ES confocal laser-scanning microscope.

### Example 5

### Immunostaining

Immunocytochemistry was performed by plating coverslips containing A549 cells cultured to 70-80% confluence. The cells were fixed with chilled methanol (Fisher Scientific, Waltham, MA) for one hour, followed by rinses with filtered PBS. Samples were blocked with 2% BSA (Fisher Scientific) in PBS containing Tween 20 (Sigma) and Triton X-100 (Sigma) for 30 minutes and incubated at room temperature on a shaker. The primary antibodies used included: IFN-γ (1:100; E-Bioscience) incubated for 2 hours on a shaker. After multiple washes in filtered PBS, coverslips were incubated with Cy5-labeled secondary antibody (1:200; Jackson ImmunoResearch, PA) for 1 hr. After four washes in PBS, cells were incubated for 5 minutes in 4′,6-diamindino-2-phenylindole (DAPI, 1:10,000; Sigma). The coverslips were mounted and dried overnight then observed under a Bio-Rad MRC1024ES confocal laser—scanning microscope, as described earlier [14].

### Example 6

### Annexin V and PI Flow Cytometry

Single cell suspensions isolated from A549 cells were stained using the dead cell apoptosis kit with Annexin V for flow cytometry (Thermofisher) according to manufacturer instructions. Cells were washed with Annexin V buffer and stained with Annexin V and PI (propidium iodide). Flow cytometry analyses were performed using the FACS Canto™ II Flow cytometer (BD Biosciences) and analyzed using FlowJo™ Software (v10). Only Annexin V, PI, and unstained cells served as control.

### Example 7

### Immunoblotting

Western Blotting was conducted using A549 cells which were seeded in a 6-well plate at a density of 0.3×106 cells/mL for 24 h and then treated with or without spike protein. After 48 h, the cells were harvested and lysed with lysis buffer containing 150 mM NaCl, 50 mM Tris (pH 8.0), 1% Triton-X, 0.1% SDS, 0.5% Na-Deoxycholate, and protease and phosphatase inhibitor cocktail to extract the total protein. The cells were transferred to microcentrifuge tubes and spun into a pellet. The supernatant was collected and analyzed for protein concentration via the Bradford method (Bio-Rad). SDS sample buffer was added to 80-100 mg total protein and boiled for 10 min. Denatured samples were electrophoresed on Novex 15% Bis-Tris gels (Life Technologies) and proteins transferred onto a nitrocellulose membrane (Bio-Rad) using the Bio-Rad Wet transfer. The membrane was washed for 10 min in PBS containing 0.1% Tween 20 (PBST) and blocked for 1 hour in Intercept blocking buffer (Li-COR). Next, membranes were incubated overnight at 4° C. under shaking conditions with the following primary antibodies; caspase-3 (1:200; Santa-Cruz), cleaved caspase-3 (1:1000; Cell-Signaling), p53 (1:200; Santa-Cruz), Bc12 (1:200; Santa-Cruz), Bad (1:200; Santa-Cruz) and β-actin (1:10,000; Abcam) was used as a loading control. The next day, membranes were washed in PBST for 30 min, and incubated with secondary antibodies (all 1:10,000; Li-Cor Biosciences) for 1 hr at room temperature, washed in PBST for 30 min and visualized under the Odyssey Infrared Imaging System (Li-COR, Lincoln, NE). Band intensities were quantified using Image J software.

### Example 8

Animals and Experimental Design: Intoxication of A/J Mice with NNK

Mice were maintained and experiments conducted in accordance with the National Institute of Health guidelines and approved by the Rush University Medical Center Institutional Animal Care and Use Committee (IACUC). A/J female mice, 6-8-week-old were obtained from Jackson Lab (Bar Harbor, ME) mice received an injection of saline containing NNK (sc-209854) (50 mg/kg body weight). The protocol was adapted from [12,15] where the negative control mice received equal volume of saline (vehicle control).

### Example 9

Treatment of NNK-Intoxicated Mice with Recombinant SARS-CoV-2 Spike S1 Protein

After 22 weeks of NNK intoxication, mice were treated with recombinant SARS-CoV-2 spike S1 intranasally at a dose of 50 ng/mouse/every other day. Recombinant spike S1 was dissolved in 4 μl normal saline, as described earlier [16] and mice were held in the supine position and 2 μl volume was delivered into each nostril using a pipet man and control mice received only normal saline.

### Example 10

### Tumor Histology

After 26 weeks of NNK intoxication, mice were euthanized with CO₂. Tumors on the surface of the lungs were counted by a person blinded to the treatment regimens followed by taking picture of the whole lungs. Then mice underwent transcardial perfusion [17]. Lungs were excised, collected and processed for histological studies. Hematoxylin-eosin (HE) staining was performed from 5 μm paraffin embedded sections to study the morphology as described in [12]. The tumor area was analyzed by Image J, and ten images from 40× fields were chosen from each group.

### Statistical Analysis

Statistical analyses were performed using Graphpad Prism 8 (GraphPad Software, Inc., La Jolla, CA). Statistical differences between means were calculated by t-test (two-tailed). Variance between multiple means were conducted via one-way ANOVA, followed by Tukey's post hoc tests. The criteria for statistical significance was p<0.05. Values are expressed as means+SD of at least three independent experiments.

### Results

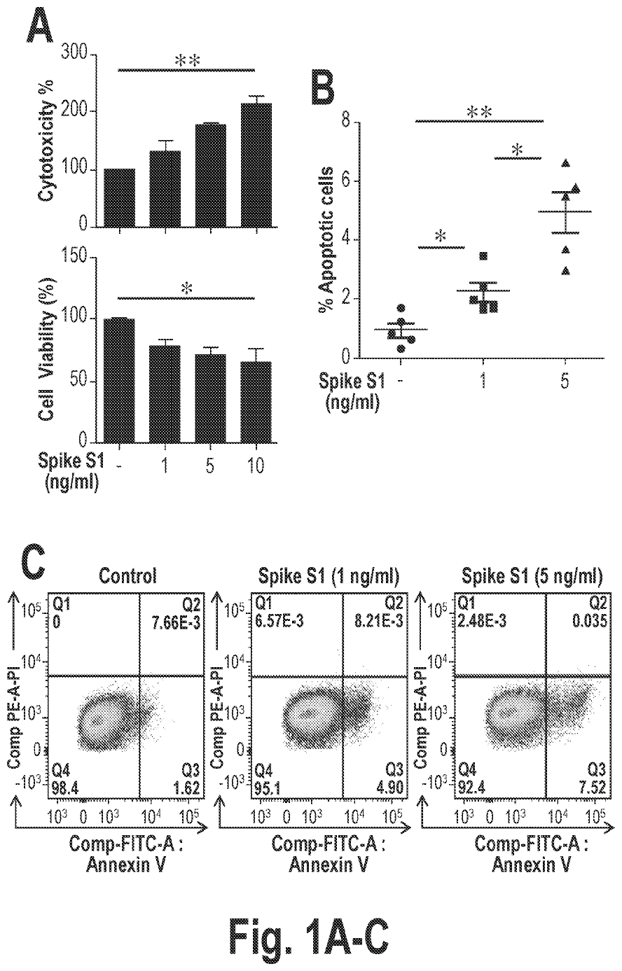

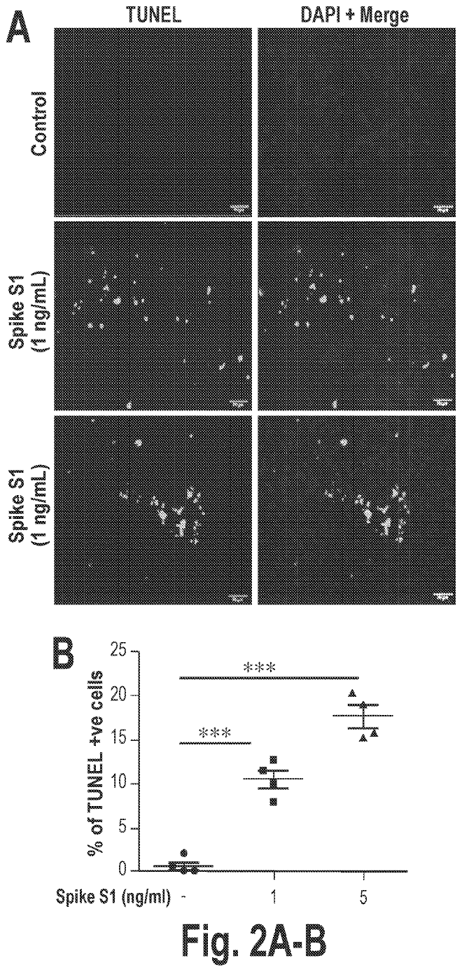

Recombinant SARS-CoV-2 spike S1 treatment induces apoptosis and death in human A549 lung cancer cells. It is commonly known that acquired resistance toward cell death is a hallmark of possibly all types of cancer [18]. Human A549 lung cancer cells were incubated with three different concentrations (1, 5, and 10 ng/mL) of recombinant Spike S1 under serum free conditions followed by measuring cell survival by LDH release and MTT assay. We found dose-dependent increase in LDH release and decrease in MTT in A549 cells by spike S1 (FIG. 1A-B). To confirm our observations, we performed dual FACS analysis with propidium iodide and annexin V (FIG. 1C-D) and found that treatment with spike S1 noticeably increased the level of apoptotic cells in A549 treated lung cancer cells. To confirm the apoptosis further, we performed TUNEL and found increase in TUNEL-positive cells by spike S1 treatment (FIG. 2A-B).

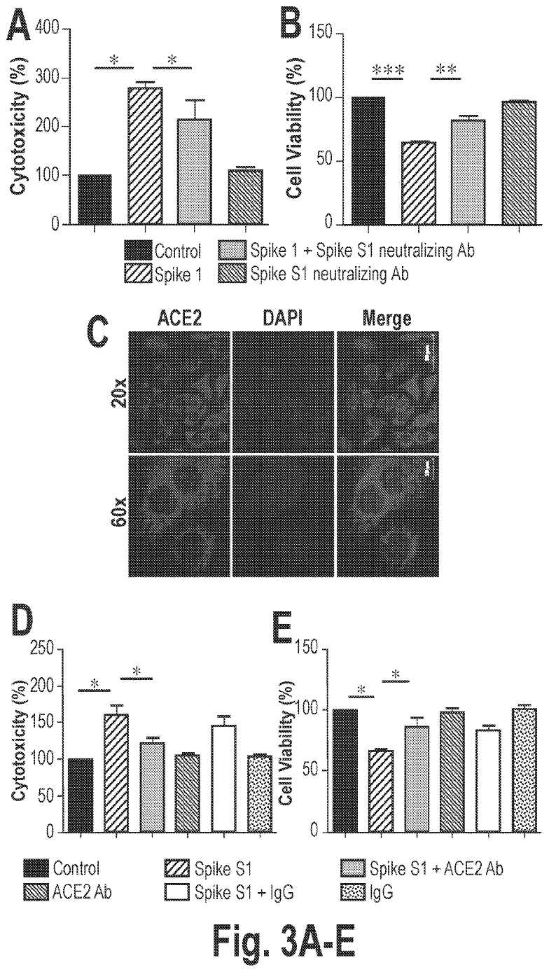

Recombinant SARS-CoV-2 spike S1 induces death of human A549 lung cancer cells via ACE2 receptor. To understand that the cell death induced by spike S1 is actually caused by spike S1, not any contaminant present with the reagent, we used neutralizing antibodies against spike S1. Suppression of spike S1-induced cell death in A549 cells by neutralizing antibodies against spike S1, but not control IgG, suggests that cell death is in fact caused by spike S1 (FIG. 3A-B).

Studies have shown that the ACE-2 receptor is expressed on the cell surface of the lung, heart, and kidneys [19]. ACE2 functions as a cellular receptor for spike S1 protein to enable viral entry into target cells. Our immunostaining results show the presence of ACE2 in A549 cells (FIG. 3C). Therefore, next, we used neutralizing antibodies against ACE2 and found inhibition of spike S1-induced death by neutralizing antibodies against ACE2, but not control IgG (FIG. 3D-E).

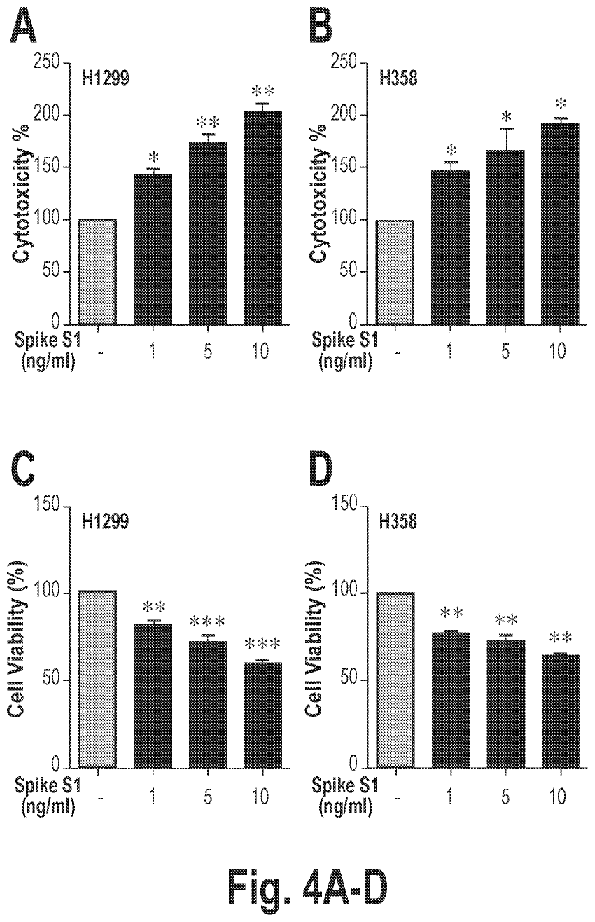

Recombinant SARS-CoV-2 spike S1 treatment leads to death of human H1299 and H358 lung cancer cells. Cancer cells are known to stave off cell death due to modifying immune surveillance [20]. We wanted to see if similar to A549 cells, spike S1 can induce death in other human lung cancer cells. We found dose-dependent increase in cell death by recombinant spike S1 in both human H1299 and H358 lung cancer cells (FIG. 4).

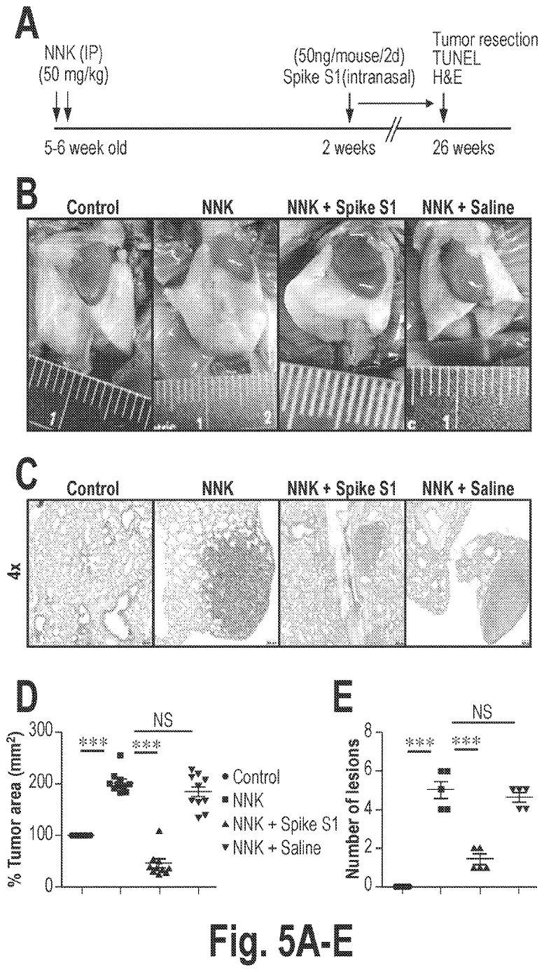

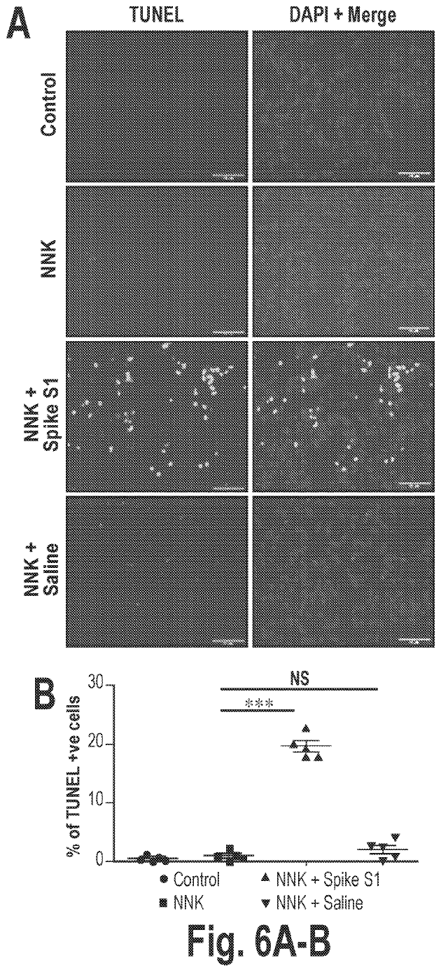

Treatment with recombinant SARS-CoV-2 spike S1 protein causes tumor regression in NNK-induced A/J mice. Studies show that A/J mice induced with 4-(methylnitrosamino)-1-(3-pyridyl)-1-butanone (NNK), a potent lung carcinogen, is a useful animal model [21]. To investigate the effects of Spike S1 protein on lung cancer tumors, we chemically induced lung carcinoma into A/J mice with NNK (FIG. 5A). Our results showed that intranasal administration of recombinant SARS-CoV-2 spike S1 protein decreased tumors in the lung of NNK-intoxicated A/J mice (FIG. 5B-D). TUNEL staining also showed that spike S1 treatment induced apoptosis in lung tumors of NNK-intoxicated A/J mice (FIG. 6A-B).

Although the foregoing disclosure has been described in some detail by way of illustration and example for purposes of clarity of understanding, it will be readily apparent to one of ordinary skill in the art in light of the teachings of this disclosure that certain changes and modifications may be made thereto without departing from the spirit or scope of the appended claims. The following examples are provided by way of illustration only and not by way of limitation. Those skilled in the art will readily recognize a variety of noncritical parameters that could be changed or modified to yield essentially similar results.

### REFERENCES

1. From the Centers for Disease Control. Chronic disease reports: deaths from lung cancer—United States, 1986. JAMA 1989, 262, 1170, 1172.
2. Dela Cruz, C. S.; Tanoue, L. T.; Matthay, R. A. Lung cancer: epidemiology, etiology, and prevention. Clin Chest Med 2011, 32, 605-644, doi:10.1016/j.ccm.2011.09.001.
3. Siegel, R. L., Miller, K. D., Jemal, A. Cancer Statistics, 2022. CA Cancer J Clin 2022, 72, 7-33, doi: https://doi.org/10.3322/caac.21708.
4. Rachow, T.; Schiffl, H.; Lang, S. M. Risk of lung cancer and renin-angiotensin blockade: a concise review. J Cancer Res Clin Oncol 2021, 147, 195-204, doi:10.1007/s00432-020-03445-x.
5. Cheng, Q.; Zhou, L.; Zhou, J.; Wan, H.; Li, Q.; Feng, Y. ACE2 overexpression inhibits acquired platinum resistance-induced tumor angiogenesis in NSCLC. Oncol Rep 2016, 36, 1403-1410, doi:10.3892/or.2016.4967.
6. Feng, Y.; Ni, L.; Wan, H.; Fan, L.; Fei, X.; Ma, Q.; Gao, B.; Xiang, Y.; Che, J.; Li, Q. Overexpression of ACE2 produces antitumor effects via inhibition of angiogenesis and tumor cell invasion in vivo and in vitro. Oncol Rep 2011, 26, 1157-1164, doi:10.3892/or.2011.1394.
7. Machhi, J.; Herskovitz, J.; Senan, A. M.; Dutta, D.; Nath, B.; Oleynikov, M. D.; Blomberg, W. R.; Meigs, D. D.; Hasan, M.; Patel, M.; et al. The Natural History, Pathobiology, and Clinical Manifestations of SARS-CoV-2 Infections. J Neuroimmune Pharmacol 2020, 15, 359-386, doi:10.1007/s 11481-020-09944-5.
8. Pahan, P.; Pahan, K. Smooth or Risky Revisit of an Old Malaria Drug for COVID-19?J Neuroimmune Pharmacol 2020, 15, 174-180, doi:10.1007/s11481-020-09923-w.
9. Paidi, R. K.; Jana, M.; Mishra, R. K.; Dutta, D.; Pahan, K. Selective Inhibition of the Interaction between SARS-CoV-2 Spike S1 and ACE2 by SPIDAR Peptide Induces Anti-Inflammatory Therapeutic Responses. J Immunol 2021, 207, 2521-2533, doi:10.4049/jimmunol.2100144.
10. Paidi, R. K.; Jana, M.; Mishra, R. K.; Dutta, D.; Raha, S.; Pahan, K. ACE-2-interacting Domain of SARS-CoV-2 (AIDS) Peptide Suppresses Inflammation to Reduce Fever and Protect Lungs and Heart in Mice: Implications for COVID-19 Therapy. J Neuroimmune Pharmacol 2021, 16, 59-70, doi:10.1007/s11481-020-09979-8.
11. Mukherjee, S.; Pahan, K. Is COVID-19 Gender-sensitive?J Neuroimmune Pharmacol 2021, 16, 38-47, doi:10.1007/s11481-020-09974-z.
12. Ito T, S. H., Guo X, Sysa-Shah P, Coulter J, Tamashiro KLK, Lee R S, Orita H, Sato K, Ishiyama S, Hulbert A, Smith W E, Peterson L A, Brock M V, Gabrielson K L. Prenatal stress enhances NNK-induced lung tumors in A/J mice. Carcinogenesis 2020, 41, 1713-1723, doi:10.1093/carcin/bgaa033.
13. Grant T. Corbett, A. R. a. K. P. Gemfibrozil, a lipid-lowering drug, upregulates interleukin-1 receptor antagonist in mouse cortical neurons: Implications for neuronal self-defense. J Immunol 2012, 189, 1002-1013, doi: https://doi/org/10.4049/jimmunol.1102624.
14. Ramesh K. Paidi, M. J., Rama K. Mishra, Debashis Dutta, and Kalipada Pahan. Selective Inhibition of the Interaction between SARS-CoV-2 Spike S1 and ACE2 by SPIDAR Peptide Induces Anti-Inflammatory Therapeutic Responses. The Journal of Immunology 2021, doi:10.4049/jimmunol.2100144.
15. Yao, Y. A deficiency in cathelicidin reduces lung tumor growth in NNK/NTHi-induced A/J mice. 2018
16. Ramesh K. Paidi, M. J., Rama K. Mishra, Debashis Dutta, Sumita Raha, and Kalipada Pahan. ACE-2-interacting Domain of SARS-CoV-2 (AIDS) Peptide Suppresses Inflammation to Reduce Fever and Protect Lungs and Heart in Mice: Implications for COVID-19 Therapy. J Neuroimmune Pharmacol 2021, doi:10.1007/s11481-020-09979-8.
17. Susanta Mondal, M. J., Sridevi Dasarathi, Avik Roy, and Kalipada Pahan. Aspirin ameliorates experimental autoimmune encephalomyelitis through interleukin-11-mediated protection of regulatory T cells. Sci Signal 2018, 11, doi:10.1126/scisignal.aar8278.
18. Weinberg, D. H. a. R. A. The hallmarks of cancer. Cell 2000, 100, 57-70, doi:https://doi.org/10.1016/S0092-8674(00)81683-9.
19. Mohammad Amin Zaman, S. O. D. A. C. Drugs targeting the renin-angiotensin-aldosterone system. Nature Reviews Drug Discovery 2002, doi:https://doi.org/10.1038/nrd873.
20. Rik Derynck, R. J. A. A. B. TGF-β signaling in tumor suppression and cancer progression. Nature Genetics 2001, 117-129, doi:https://doi.org/10.1038/ng1001-117

1. A method of treating cancer comprising administering to a patient in need thereof, a therapeutically effective amount of a recombinant SARS-CoV-2 Spike S1 or a biologically active fragment thereof.

2. The method of claim 2 wherein the recombinant SARS-CoV-2 Spike S1 or a biologically active fragment thereof comprises the amino acid sequence of SEQ ID NO. 1.

3. The method of claim 1, wherein the recombinant SARS-CoV-2 Spike S1 or a biologically active fragment thereof is administered intranasally, subcutaneously, or intravenously.

4. The method of claim 1, wherein the recombinant SARS-CoV-2 Spike S1 or a biologically active fragment thereof is administered intranasally

5. The method of claim 1, wherein the therapeutically effective amount comprises about 1 to about 50 ng/mL.

6. The method of claim 1, wherein the therapeutically effective amount comprises about 50 ng/kg body weight.

7. The method of claim 1, wherein the therapeutically effective amount comprises about 1 ng/mL.

8. The method of claim 1, wherein the therapeutically effective amount comprises about 5 ng/mL.

9. The method of claim 1, wherein the therapeutically effective amount comprises about 10 ng/mL.

10. The method of claim 1, wherein the cancer comprises lung cancer, kidney cancer or heart cancer.

11. The method of claim 10, wherein the cancer comprises lung cancer.

12. The method of claim 1, further comprising administration of one or more additional therapeutic agents.

13. An isolated SARS-CoV-2 Spike S1 polypeptide comprising the amino acid sequence of SEQ ID NO: 1.

14. A pharmaceutical composition comprising the polypeptide of claim 14 and a pharmaceutically acceptable carrier.
